(19) United States
(12) Patent Application Publication
(10) Pub. No.: US 2025/0344994 A1
(43) Pub. Date: Nov. 13, 2025

(54) METHOD AND SYSTEM TO MONITOR URINE OUTPUT AND MANAGE FLUID RETENTION IN A PATIENT

(71) Applicant: REPRIEVE CARDIOVASCULAR, INC., Milford, MA (US)

(72) Inventors: Howard LEVIN, Teaneck, NJ (US); Andrew V. HALPERT, Brookline, MA (US); Mark GELFAND, New York, NY (US)

(21) Appl. No.: 19/169,270

(22) Filed: Apr. 3, 2025

Related U.S. Application Data
(63) Continuation of application No. 16/544,975, filed on Aug. 20, 2019, now Pat. No. 12,290,380.
(60) Provisional application No. 62/720,009, filed on Aug. 20, 2018.

(51) Int. Cl. A61B 5/00; A61B 5/0295; A61B 5/20; A61M 5/172; A61M 25/00

(52) U.S. Cl. CPC A61B 5/4839; A61B 5/0295; A61B 5/208; A61M 5/1723; A61M 25/0017; A61M 2202/0496

(57) ABSTRACT

A method to treat patients suffering from fluid overload including: administrating a diuretic to the patient to increase urine output of the patient; monitoring intravascular volume of the patient; and maintaining the patient in a condition in which the intravascular volume is below a baseline intravascular volume and above a hemodynamic level by adjusting the administered diuretic.

### CROSS-REFERENCE TO RELATED APPLICATION(S)

This application is a continuation of U.S. patent application Ser. No. 16/544,975, filed Aug. 20, 2019, which claims priority to U.S. provisional application 62/720,009, filed Aug. 20, 2018, the entireties of which are incorporated by reference.

### TECHNICAL FIELD

The field of the invention is monitoring urine output and managing a patient's fluid levels using a diuretic.

### BACKGROUND

The body of a healthy person naturally maintains a healthy balance of fluids. Patients often do not naturally achieve fluid balance when suffering from acute decompensated heart failure (ADHF), acute kidney injury (AKI) and other similar conditions. The kidneys in these patients are unable to generate sufficient urine to avoid fluid overload and maintain a proper fluid balance. A fluid overloaded patient is treated to reduce the fluid volume in the body, such as by receiving diuretics which promote urine output. Generally, fluid volume should be reduced in a rapid, safe and effective manner to reduce fluid levels (decongestion) in the patient.

Liquids normally represent about half to three-quarters of the body weight of a health person. Approximately two-thirds of the total liquid in a body are in the cells of the body (intracellular). The remaining one-third of total liquids are outside of the cells (extracellular). Most of the extracellular liquid bathes the cells (interstitial liquid). Another portion of extracellular liquid is in blood and circulates through the vascular system. The portion of total body liquid in the blood is typically about 7% of total body liquids. Wikipedia, Extracellular Fluid (Aug. 17, 2018). The body removes excess liquid primarily by generating urine. Secondary modes for removing liquids are by evaporation through skin such as sweating, discharge in feces, and evaporation from the lungs.

Artificially promoting urine production by administration of a diuretic is a well-known and standard practice for reducing fluid levels in a patient. The choice of type, amount and timing of diuretics affect the amount of urine that is produced and thus the rate at which fluid is removed from the body. Diuretics may be introduced by an intravenous (IV) line. If treatments with diuretics are unsuccessful, ultra-filtration may be used to reduce fluid levels to, for example, treat ADFH.

The short-term effects of diuretic administration on urine production for an individual are not entirely predictable. In response to a dose of a diuretic, a patient may produce much less urine than expected which may prolong a hospital stay or cause an outpatient to be hospitalized. Another patient may response to a dose of diuretic by producing excessive amounts of urine which raises concerns hypotension and vital organ damage.

The potential for substantially different responses and treatment outcomes in response to a dosage of diuretics creates uncertainties for physicians who have to determine correct diuretic dosing for an individual patient based on the patient's clinical signs and symptoms. Physicians may prescribe a conservative (low) diuretic dosage and later slowly increase the dose to achieve a desired urine output. This conservative approach can prolong the treatment and may render the patient unable to produce sufficient amounts of urine. Disadvantages of the conservative approach are that the patient's symptoms may be prolonged and the underlying clinical state may worsen due to the slow application of diuretics.

There is a long felt need to provide physicians with diagnostic information and recommended treatments for patients in a fluid overload condition and being treated to reduce the fluid volume in their body.

### SUMMARY

The inventors conceived of and disclose herein a novel method of treatment to reduce fluid levels in a fluid overloaded patient, such as one suffering from ADHF, AKI or other condition that results in fluid overload. The method including a regimen that: (i) determines whether the patient is suited for diuretic therapy, (ii) rapidly reduces the intravascular volume of fluid in the patient to a range below a threshold low level of intravascular volume and a hemodynamic level below which there is a risk of vital organ damage due to low intravascular volume, and (iii) maintains the intravascular volume in the range to allow extravascular fluid in the body to move into the vasculature and be removed by the kidneys generating urine.

The inventors invented a method to treat patients suffering from fluid overload including: administrating a diuretic to the patient to increase urine output of the patient; monitoring intravascular volume of the patient; and maintaining the patient in a condition in which the intravascular volume is below a baseline intravascular volume and above a hemodynamic level by adjusting the administered diuretic.

A physician prescribes diuretics that are administered to the patient to promote urine output. The physician selects the diuretic, dosage and timing of the administration of the dosage to achieve urine output that rapidly reduces intravascular volume and subsequently maintains intravascular volume in the range.

A fluid management device, such as the RenalGuard® system may be used to monitor urine output and promote urine output by injecting a hydration fluid into the patient and monitoring urine output. The injection of the fluid is controlled by monitoring urine output and using urine output as feedback to control the amount of fluid added to the patient. The fluid management device controls the amount of fluid added to the patient such that there is a net reduction in the amount of fluid in the patient. The fluid management device may also detect if the patient is not producing sufficient amounts of urine in response to diuretics and then automatically stops fluid injection and issues an alert suggesting that other treatments, such as ultrafiltration, may be appropriate.

The information provided by the urine monitoring device may be used to determine if a fluid overloaded patient should be treated with diuretics to promote urine output, whether administered diuretics are causing the kidneys to generate urine, whether a dosage of diuretics should be adjusted, whether a fluid should be infused into the patient to promote kidney functioning or otherwise assist the patient, and whether the patient is becoming hypotensive or otherwise suffer problems associated with unnaturally low intravascular fluid levels.

The invention may also be embodied as a method to reduce liquid levels of a patient comprising: determining a maximum liquid loss limit for the patient; monitoring urine output by the patient; infusing a hydration liquid into the patient; determining a net rate of liquid removal from the patient based on a difference between the urine output and the infusion of the hydration liquid, and automatically adjusting the infusion of the hydration liquid to maintain the net rate of liquid removal below the maximum liquid loss limit.

The maximum liquid loss limit may be a maximum net rate of liquid lost by the patient. The infusion of the hydration liquid and the automatic adjusting of the infusion of the hydration liquid may be performed using a hydration liquid system including an infusion controller configured to control a pump configured to pump the hydration liquid into the patient.

The infusion of the hydration liquid into the patient may initially be at a high rate which is greater than the urine output, and the high rate is late reduced to below the urine output. The high rate of hydration fluid may be reduced in response to the urine output exceeding a predetermined high threshold rate.

The infusion of the hydration liquid at the high rate may be performed by automatically infusing the hydration liquid at a high rate greater than a concurrent rate of urine output while the concurrent rate of urine output is below a threshold urine rate, and automatically reducing the high rate of the hydration liquid to below the concurrent rate of urine output in response to the concurrent rate of the urine output exceeding the threshold urine rate.

The invention may also be embodied as a method to reduce liquid levels of a patient comprising: repeatedly determining a current urine rate by the patient; initially infusing a hydration liquid into the patient at a high hydration rate greater than the current urine rate; and reducing rate of infusion of the hydration liquid to a rate below the current urine rate in response to the current urine rate exceeding a predetermined high threshold rate.

The determination of the current urine rate may be performed at intervals of no greater than five minutes.

The method may include administering a diuretic to the patient while infusing the hydration liquid at the high hydration rate.

The method may further include: determining a net rate of liquid removal from the patient based on a difference between the urine output rate and a rate of the infusion of the hydration liquid, and automatically adjusting the infusion of the hydration liquid to maintain the net rate of liquid removal below a maximum liquid loss limit.

### BRIEF DESCRIPTION OF THE DRAWINGS

The following figures illustrate the invention and is use:

FIG. 1 is a schematic view of one embodiment of a patient hydration system which is configured to monitor urine output and control the injection of a fluid into a patient.

FIG. 2 is a graphical representation of a regimen for treating fluid overload.

FIGS. 3 and 4 are a flow chart of an exemplary optimization phase of the regimen.

### DETAILED DESCRIPTION

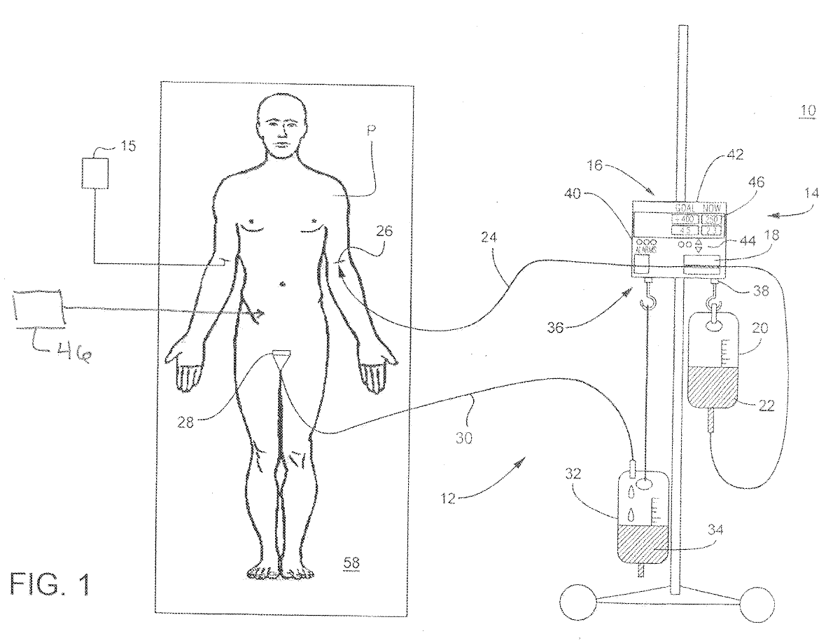

FIG. 1 shows a patient fluid management system 10 that includes a urine collection system 12 and an optional hydration fluid infusion system 14 both of which are connected to patient P. The patient is suffering from fluid overload and may be hospitalized and receiving diuretics 15 through an intravenous (IV) line. The diuretics may be added to a line injecting the hydration fluid or may be added to another saline solution filled bag 15 connected to the patient via another IV line.

The optional hydration fluid infusion system 14 includes an infusion controller 16, that includes an infusion pump 18, e.g., a peristaltic pump, connected to a fluid source 20, e.g., saline bag, of an hydration fluid 22, e.g. saline, by tubing (line) 24. An intravenous (I.V.) needle 26 is inserted in a vein of the patient P and is connected to infusion pump 18 via tubing 24. Fluid 22 from the source 20 flows through the tubing 24 and I.V. needle 26 directly into a blood vessel, e.g., peripheral vein, of the patient P. The amount or rate of fluid(s) 22 flowing into the patient may be determined by the pumping rate or number of rotations the infusion pump 18.

The urine collection system 12 includes a catheter 28, such as a Foley catheter, placed in the bladder of patient P. Tubing 30 connects catheter 28 to a urine collection device, such as a bag 32. The urine 34 collected in the bag 32 is weighed or otherwise measured by a weight scale 36 or other urine flow measurement device which communicates with the infusion controller 16. A weight scale 38 may also weight the hydration fluid 22.

The amount or rate of urine 34 is monitored in real time by the infusion controller 16. Similarly, the amount of hydration fluid 22 in the fluid source 20 may be monitored or measured by a weight scale 38. The weight scales 36, 38 may be a single weight scale which measures the combined change in urine output and fluid input by and to the patient. The combined change in urine output and fluid input indicates the net fluid loss or gain by the patient.

The infusion controller 16 monitors the weight of the hydration fluid 22, the amount of the hydration fluid 22 pumped through pump 18 or otherwise monitors, in real time, the amount or rate of hydration fluid 22 flowing into the patient P.

The fluid management system 10 may be the RenalGuard System®, developed and marketed by RenalGuard Solutions, Inc. of Milford, Massachusetts, which in the past has been used to protect patients from kidney injury during procedures that require iodinated contrast agents.

A computer control system 40 in the infusion controller 16 receives an input as to a desired negative fluid balance, and/or amount(s) or rate(s) of urine output and/or of a desired amount(s) or rate(s) of a difference urine output and the amount of hydration fluid. A negative fluid balance refers to injecting less hydration fluid 22, in terms of mass or flow rate, into the patient than the amount of urine 34 output. The fluid balance may be repeatedly determined, such as every thirty minutes, every hour or every few hours. During the treatment period, the amount of hydration fluid injected into the patient may initially be greater than the amount of urine output, in an effort to start a high urine output flow. Later in the treatment period, such as after the urine output flow reaches a predetermined high threshold rate, the rate of infusion of the hydration fluid may be reduced. The high urine output flow is expected to continue after the reduction of the rate of the hydration fluid.

The computer control system 40 may include a processor(s) and a non-transient memory configured to store program instructions, settings for the patient fluid management system 10 and data collected from or calculated by the computer control system 40. The data may include urine output volume or rate of urine output, amount of fluid infused into the patient and rate of infusion, the amount and rate of injection of a diuretic, the weight of the patient at various times during the infusion of the fluid, and the time during which the patient is treated with the patient fluid management system 10. The computer control system 40 may include a console 42 having a user input device 44, such as a key pad, and a user output device 46, such as a computer display.

The input device 44 may be used to input certain parameters of the treatment sessions, such as a desired fluid balance level, desired urine output level, and the planned duration of the input balance level or urine output level. Another input may be the amount of fluids during the treatment session received by the patient through means other than the fluid source 20. For example, the input device 44 may be configured to receive inputs indicating the amount of fluid included with a saline filled bag 15 used to inject the diuretic into the patient.

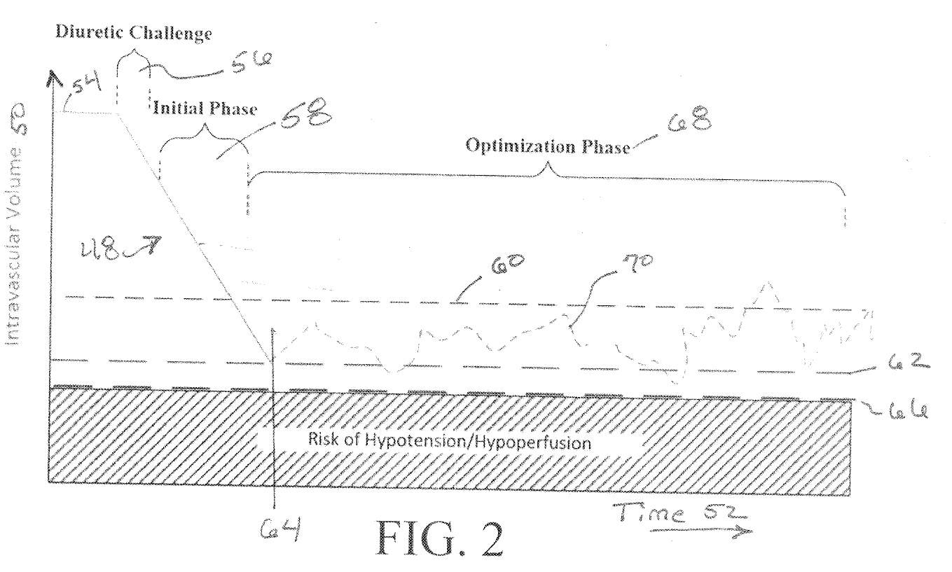

FIG. 2 is a diagram illustrating an exemplarily regimen to reduce fluid levels in a patient suffering from fluid overload. The diagram shows the relationship between a desired target 48 intravascular volume (horizontal axis 50) and time (vertical axis 52). The intravascular volume is the fluid (blood plasma) in the vasculature of the body.

Intravascular volume is used in FIG. 2 to illustrate a desired therapeutic effect of administering a diuretic and promoting urine output. Intravascular volume is measured using a system 46 that measures a parameter of the patient from which intravascular volume can be calculated or estimated. Moreover, other parameters from which intravascular volume or a change in such volume may be calculated or estimated include: measuring urine output, calculating net fluid loss (urine output minus fluid intake); measuring central venous pressure (CVP), measuring renal perfusion pressure, and measurements of concentrations of chemicals in urine such as the partial pressure of oxygen in urine, potassium, sodium and keratein.

A patient in a fluid overloaded condition as indicated by an excessive intravascular fluid level 54 is a candidate for the regimen shown in FIG. 2. During an initial phase 56 of the regimen, an evaluation is made as to whether the patient sufficiently responds to a diuretic. An initial dose of a diuretic may be administered, and the urine output is monitored to determine if there is sufficient increase in urine output rate in response to the diuretic. For example, a goal of having the patient excrete at least 200 ml of urine within four (4) hours of receiving a dose of a diuretic may be used to determine if the patient is suited for proceeding beyond the diuretic challenge 56 and to the initial phase 58.

If the patient is responsive to a diuretic to promote urine output, a dose of a diuretic is applied to cause a high urine output during an initial phase 58 of the regimen. During the initial phase, urine output is increased to cause the intravascular volume 48 to fall below the normal base line 60 for intravascular volume and to a target minimum level 62 for intravascular volume.

The current level of intravascular volume is determined based measurements of blood pressure, cardiac output and/or systemic vascular resistance. The measurements may be preformed by a system 46 configured to measure one or more of blood pressure, cardiac output and systemic vascular resistance. These measurements may be preformed continuously or at regular intervals, such as every 10 minutes, 30 minutes or hourly. An example of a monitoring system to sense parameters indicative of intravascular volume and/or changes in intravascular volume is an intraintrathoracic impedance monitoring (such as may included in be included in an implanted defibrillator or biventiricular pacing device);

During the initial phase, the rate of net fluid volume removal may be controlled by the fluid management system to be at a fluid loss limit (FLL) rate or at a rate offset by a selected safeguard for the FLL. The FLL is a maximum rate of net volume removal the fluid management system will target. The FLL may be defined clinically as a rate which the physician determines as rate of net amount of fluid volume removal from the patient without adverse hemodynamic changes or activation of central SNS or renal salt and water retaining mechanisms.

The fluid management system may automatically infuse the hydration fluid to ensure that the FLL is not exceeded. For example, if the kidneys are producing urine at a rate of 500 ml/hour and the FLL is set to a rate of 200 ml/hour, the system will infuse replacement fluid at a rate of 300 ml/hour. If urine production is below the FLL rate, the fluid management system may not infuse a hydration fluid or may infuse the hydration fluid at a rate intended to ensure that renal perfusion pressure is sufficient to promote generation of urine by the kidneys.

The base line is the amount of intravascular volume (blood plasma) that the patient would have when healthy and properly functioning kidneys.

The target minimum intravascular level 62 is defined clinically as the minimum level of intravascular volume that can maintain sufficient cardiac output to allow adequate renal perfusion. Alternatively, the target minimum intravascular level 62 may be defined physiologically as a 10% decrease in cardiac output as compared to the cardiac output at the base line 60 and/or a 10% increase in systemic vascular resistance from that at the baseline (60).

The rapid reduction in intravascular volume is intended to stop at time 62 which is when the intravascular volume reaches the target minimum intravascular level. The reduction in intravascular volume may be achieved by reduction in the type or dosage of the diuretic and/or by increasing the infusion rate of a hydration fluid 22. The infusion controller 16 may monitor in real time or by receiving user inputs an indicator(s) of intravascular volume and automatically increase (or start) the infusion rate of the hydration fluid. Similarly, the physician may manually increase the rate of the infusion fluid and adjust the type and/or dosage of the diuretic to slow or halt the reduction in intravascular fluid.

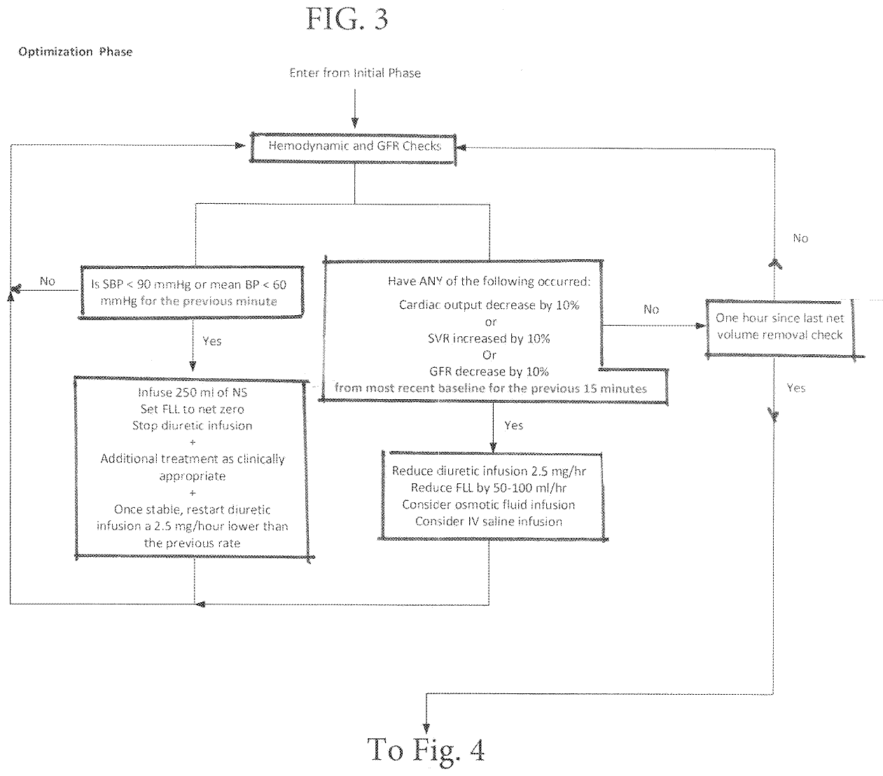

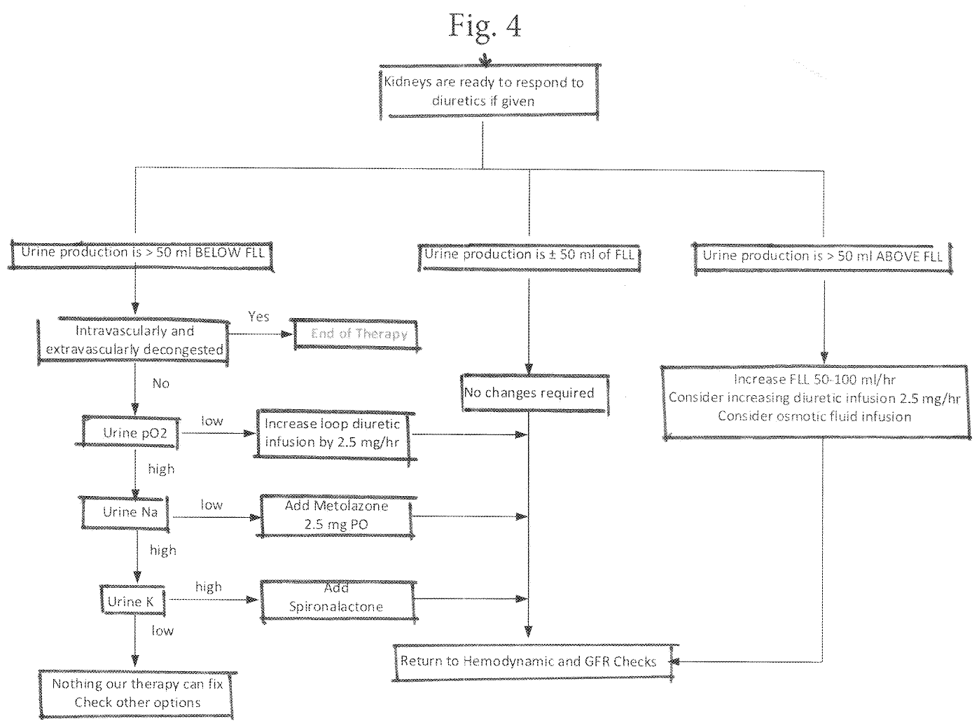

As shown in FIGS. 3 and 4, the optimization phase 68 of the regimen maintains the current intravascular volume 70 (FIG. 2) between the baseline intravascular volume 60 and the hemodynamic level 66. The optimization phase operates to keep the current intravascular volume 70 at the target minimum intravascular volume 62.

During the initial phase, it is believed that the liquid in the vascular system is reduced as the kidneys produce urine. Other liquids in the body (interstitial and intracellular liquids) are slow to move into the vasculature. During at least an early portion of the initial phase, the interstitial and intracellular liquids are not flowing rapidly into the vasculature and thus the amount of liquid in the vascular volume drops rapidly as the urine rate is increased due to the diuretic. Moreover, the flow of interstitial and intracellular fluids into the vascular volume may remain at or below a maximum urine rate that can be achieved with aggressive dosages of a diuretic. Thus, during the optimization phase 68, the dosage of the diuretic and the infusion of the hydration fluid may be controlled to cause a urine rate that is commiserate, e.g., approximately equal too, the predicted rate at which interstitial and intracellular fluids enter the vascular volume.

During an optimization phase 68, the current intravascular volume level 70 moves above and below the target level 62. During this phase, the patient remains under treatment by a diuretic. Also, the fluid management system adjusts the infusion of the hydration fluid to achieve desired levels of urine output and negative fluid balance in the patient. During the optimization phase 68, the PRR is expected to be the high end at or slightly above a predefined range, such as 100 to 300 ml/hour.

During the optimization phase, the physician prescribes a diuretic and dosage which is intended to maintain the current intravascular volume level 70 at the target minimum intravascular volume 62. If the current intravascular volume level 70 is consistently below the target 62, the patient may be in an over-diuresis condition due to excessive urine output as compared to the rate at which interstitial and intracellular fluids move into the vascular volume to replace liquid (blood plasma) removed from the vascular volume due to urine. A patient that is in an over-diuresis condition may be a candidate to have a lower dosage of the diuretic or another less aggressive diuretic.

If the current intravascular volume level 70 is consistently above the target, the patient may be in an under-diuresis condition in which the urine output is insufficient to maintain the target 62 vascular volume. A patient that is in an under diuresis condition may be a candidate to receive a higher dosage of diuretic or a more aggressive diuretic.

Adjusting the diuretic type/dosage and/or the infusion of a hydration fluid is used to keep the intravascular volume 48 above a level 66 of an at-risk intravascular volume level. The at-risk intravascular volume level is a minimum volume below which the patient is at risk for vital organ damage, and/or the systolic arterial blood pressure is less than 90 mmHg and/or a mean arterial blood pressure is less than 60 mmHg. The intention is to maintain the intravascular fluid level always above the level 66 at which there is a risk of hypotension and/or hypoperfusion. In a fluid overload patient, there are excessive body liquids (interstitial and intracellular) beyond the liquids in the intravascular volume. These excess liquids should be removed to treat the fluid overload condition.

The regimen proposed in FIG. 2 removes these other excessive body liquids (interstitial and intracellular) by holding the intravascular volume at levels below baseline 60 and at or near a target level 62. Because the intravascular volume is well below the baseline 60, the remaining body liquids will tend to more rapidly flow into the vasculature as the body attempts to increase the vascular volume to at least the base line. The flow of the remaining body fluids into the vascular volume is the plasma refilling rate (PRR).

The fluid management system 14 monitors the net removal of fluids from the body, such as by measuring the urine output and the hydration liquid input (and possibly inputs from other liquids). The fluid management system may produce alerts or reports that the patient is in an under-diuresis or over-diuresis condition. The physician, with the assistance of the fluid management system, may modify the diuretic or its dosage to treat the under-diuresis or will continue the regimen until a desired fluid balance is achieved in the patient.

The fluid management system 14 may also automatically adjust the pumping rate of the hydration fluid or generate a report of suggested pumping rates. The fluid management system 14 may automatically increase the pumping rate of the hydration fluid in response to a determination that the current intravascular volume 70 is below the target minimum 62. The amount of increase in the rate of the hydration fluid may be proportional to the difference between the current intravascular volume 70 and the target minimum 62 or proportional to a difference between the current intravascular volume 70 and the at-risk intravascular volume level 66.

The optimization phase 68 continues until the patient has been decongested such that the total body fluid is within a desired range.

Although specific features of the invention are shown in some drawings and not in others, this is for convenience only as each feature may be combined with any or all of the other features in accordance with the invention. For example, there are other ways to determine a patient's urine output and other ways to quantify the amount of hydration fluid administered to the patient. There are also other ways to redundantly check the amount of hydration fluid administered the patient. Also, the words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

1-19. (canceled)

20. A method to reduce liquid levels of a patient, the method comprising:
obtaining an intravascular fluid volume of a patient;
obtaining an intravascular fluid volume threshold for the patient;
during a first period, causing a hydration liquid to be administered to the patient at a first hydration rate to reduce the intravascular fluid volume of the patient; and
during a second period after a urine output rate of the patient reaches or exceeds a predetermined urine output threshold, causing the hydration liquid to be administered to the patient at a second hydration rate different than the first hydration rate and less than the urine output rate, wherein, during the second period, the intravascular fluid volume of the patient is above the intravascular fluid volume threshold.

21. The method of claim 20, further comprising:
obtaining a maximum rate of net volume removal for the patient,
wherein causing the hydration liquid to be administered to the patient at the first hydration rate includes causing a difference between the urine output rate and the first hydration rate to be less than the maximum rate of net volume removal.

22. The method of claim 21, wherein the maximum rate of net volume removal is greater than a net volume removal rate associated with one or more adverse hemodynamic changes.

23. The method of claim 21, wherein:
the maximum rate of net volume removal is a first maximum rate of net volume removal, and
the method further comprises, when the urine output rate during the first period is greater than the maximum rate of net volume removal, obtaining a second maximum rate of net volume removal, wherein the second maximum rate of net volume removal is greater than the first maximum rate of net volume removal.

24. The method of claim 20, further comprising:
causing an initial dose of a diuretic to be administered to the patient; and
in response to an increase to the patient's urine output following the initial dose, causing an additional dose of the diuretic or another diuretic to be administered to the patient.

25. The method of claim 20, further comprising:
causing an initial dose of a diuretic to be administered to the patient; and
after the initial dose, causing an additional dose of the diuretic or another diuretic to be administered to the patient while the intravascular fluid volume of the patient is decreasing.

26. The method of claim 20, wherein obtaining the intravascular fluid volume includes obtaining the intravascular fluid volume based at least partially on a concentration of sodium in urine obtained from the patient.

27. The method of claim 20, wherein obtaining the intravascular fluid volume includes obtaining the intravascular fluid volume based at least partially on a partial pressure of oxygen in urine obtained from the patient.

28. The method of claim 20, further comprising, during the second period:
determining that the intravascular fluid volume of the patient is increasing, and
adjusting the second hydration rate and/or a dosage of a diuretic to cause a decrease to the intravascular fluid volume.

29. The method of claim 20, wherein:
the intravascular fluid volume threshold is a minimum intravascular fluid volume threshold,
causing the hydration liquid to be administered to the patient at the first hydration rate includes causing the intravascular fluid volume of the patient to be reduced to a target intravascular fluid volume greater than the minimum intravascular fluid volume threshold, and
causing the hydration liquid to be administered to the patient at the second hydration rate occurs while the intravascular fluid volume increases from the target intravascular fluid volume toward a baseline intravascular volume.

30. A system for reducing liquid levels in a patient, the system comprising:
a measurement device configured to obtain data associated with an intravascular fluid volume of a patient;
a hydration liquid source containing a hydration liquid;
a processor operably coupled to the measurement device; and
a non-transitory, computer-readable medium carrying instructions that, when executed by the processor, cause the processor to perform operations comprising:
determining, based at least partially on the data obtained via the measurement device, the intravascular fluid volume of the patient;
obtaining an intravascular fluid volume threshold of the patient;
during a first period, causing the hydration liquid to be administered to the patient at a first hydration rate to reduce the intravascular fluid volume of the patient; and
during a second period after a urine output rate of the patient reaches or exceeds a predetermined urine output threshold, causing the hydration liquid to be administered to the patient at a second hydration rate different than the first hydration rate and less than the urine output rate, wherein, during the second period, the intravascular fluid volume of the patient is above the intravascular fluid volume threshold.

31. The system of claim 30, wherein:
the operations further comprise obtaining a maximum rate of net volume removal for the patient, and
causing the hydration liquid to be administered to the patient at the first hydration rate during the first period includes causing a difference between the urine output rate and the first hydration rate to be greater than the maximum rate of net volume removal.

32. The system of claim 31, wherein the maximum rate of net volume removal is greater than a net volume removal rate associated with one or more adverse hemodynamic changes.

33. The system of claim 31, wherein:
the maximum rate of net volume removal is a first maximum rate of net volume removal, and
the operations further comprise, when the urine output rate during the first period is greater than the maximum rate of net volume removal, obtaining a second maximum rate of net volume removal, wherein the second maximum rate of net volume removal is greater than the first maximum rate of net volume removal.

34. The system of claim 30, wherein the operations further comprise:
causing an initial dose of a diuretic to be administered to the patient; and
in response to an increase to urine output by the patient following the initial dose, causing an additional dose of the diuretic or another diuretic to be administered to the patient.

35. The system of claim 30, wherein the operations further comprise:
causing an initial dose of a diuretic to be administered to the patient; and
after the initial dose, causing an additional dose of the diuretic or another diuretic to be administered to the patient while the intravascular fluid volume of the patient is decreasing.

36. The system of claim 30, wherein the measurement device is configured to obtain a concentration of sodium in urine obtained from the patient and wherein the operations comprise obtaining the intravascular fluid volume of the patient based at least partially on the obtained concentration of sodium in urine.

37. The system of claim 30, wherein the measurement device is configured to obtain a partial pressure of oxygen in urine obtained from the patient and wherein the operations comprise obtaining the intravascular fluid volume of the patient based at least partially on the obtained partial pressure of oxygen in urine.

38. The system of claim 30, wherein the operations further comprise, during the second period:
determining that the intravascular fluid volume of the patient is increasing, and
adjusting the second hydration rate and/or a dosage of a diuretic to cause a decrease to the intravascular fluid volume.

39. The system of claim 30, wherein the intravascular fluid volume threshold is a minimum intravascular fluid volume threshold and wherein the operations further comprise:
causing the hydration liquid to be administered to the patient at the first hydration rate includes causing the intravascular fluid volume of the patient to be reduced to a target intravascular fluid volume greater than the minimum intravascular fluid volume threshold, and
causing the hydration liquid to be administered to the patient at the second hydration rate occurs while the intravascular fluid volume increases from the target intravascular fluid volume toward a baseline intravascular volume.
